(19) United States
(12) Patent Application Publication
(10) Pub. No.: US 2005/0081864 A1
(43) Pub. Date: Apr. 21, 2005

(54) Ultra sheer natural latex insertable condom with ribbing and snap holes

(76) Inventor: Jerry Allen Carnevale, Los Angeles, CA (US)

Correspondence Address: CHRISTIE, PARKER & HALE, LLP, PO BOX 7068, PASADENA, CA, 91109-7068, US

(21) Appl. No.: 10/928,661

(22) Filed: Aug. 27, 2004

Related U.S. Application Data
(60) Provisional application No. 60/498,675, filed on Aug. 28, 2003.
(60) Provisional application No. 60/584,646, filed on Jul. 1, 2004.

(51) Int. Cl. A61F 6/06; A61F 6/04

(52) U.S. Cl. 128/844

(57) ABSTRACT

An insertable condom comprises a sheath of material formed to enclose a volume leaving an opening at one end, a ribbed member connected to the sheath of material, and an anchor point attached to the ribbed member. A wearable prophylactic system may be employed with the insertable condom, said system comprising a garment having a strap with an engagement device. A method for manufacturing a prophylactic device comprises forming a first portion of a prophylactic device using a poured molding process and subjecting the first portion of the prophylactic device to a dip molding process to add a second portion to the prophylactic device.

### CROSS-REFERENCE TO RELATED APPLICATION

This application is related to and claims priority from the provisional Application No. 60/498,675 filed Aug. 28, 2003, and the provisional Application No. 60/584,646 filed Jul. 1, 2004.

### FIELD OF THE INVENTION

The present invention relates to a prophylactic device. More specifically, the present invention relates to a prophylactic which is integral with an undergarment, such as a pair of panties, which undergarment may be worn in normal use as well as for sexual activity.

### BACKGROUND

Condoms are widely known in the art as a means of providing a mechanical barrier for preventing the transmission of fluids during intercourse. Condoms are a safe and reliable fertility regulation method fully under the control of the user, are not systemic in action, protect both parties against the transmission of sexually transmitted diseases (STDs), and need not be provided by a health service. A common type of condom is made to fit over the male penis during intercourse. However, this type must be put on just before intercourse begins and sexual partners may not wish to interrupt their sexual activity to put on such a device, nor may the device be readily accessible at that time.

An alternative design exists wherein a condom is designed to be inserted into and remain within the vaginal canal during intercourse. Because the insertable condom may be placed well in advance of a sexual encounter, it compares favorably with the male condom in that it does not interrupt sexual activity. Furthermore, the insertable condom is not dependent on the male erection, so it will not interfere with sexual spontaneity.

Users find that the insertable condom is an empowering and positive additional choice in the available contraceptive method mix. The insertable condom, also known as the female condom, is a thin and strong sheath of material that loosely lines the inside of a wearer's vagina. A soft flexible plastic ring may be placed at each end to help hold the device in place. Placed securely within the vagina, insertable condoms act as a mechanical barrier to help prevent pregnancy and STDs by protecting the vagina, cervix and external genitalia, affording extensive barrier protection. These insertable condoms have entered the market as an alternative for female users who want to be able to use some sort of barrier birth control method where classical female barrier methods are not sufficiently effective (e.g. the diaphragm), or for users seeking a more convenient form of STD protection.

An insertable condom is taught in U.S. Pat. No. 4,735,621 to Hessel including an outer ring to keep the condom in place over the vaginal entrance. However with the design of Hessel, despite the use of the outer ring, the condom may be pushed wholly into the vagina during intercourse allowing the penis to penetrate the vagina in an unprotected manner. Furthermore, incorrect use may lead to the penis slipping around the edge of the outer ring, and penetrating between the vaginal wall and the outside of the condom, thus defeating the purpose of the device. An improved condom is needed which addresses one or more of these failures of the prior art.

### SUMMARY OF THE INVENTION

In one embodiment, the following invention is related generally to a female or insertable condom barrier contraceptive. In an alternative embodiment, the invention is related to a combination insertable condom and thong device. In an exemplary embodiment, the thong is a feminine thong-type undergarment to which a releasable, securable, and insertable condom can be attached and thereafter worn before and during intercourse. The thong is easy to use, sensual and attractive in appearance, and can be made at a low cost for mass production. The thong with an attached insertable condom is an easily used system that minimizes shifting of the insertable condom, keeping it in place.

The condom itself provides a barrier to the entrance of male sperm and bodily fluids which may cause pregnancy or STDs such as AIDS. The insertable condom is placed in the wearer's vaginal opening and the basal aperture is connected to the thong device. In one embodiment, the thong has a plurality of attached, adjustable, securable and stretchable fabric straps incorporating engagement devices so that the straps may be secured both to each other, and/or to the condom device. In an alternative embodiment, the thong includes four fabric straps in two pairs. In a further alternative embodiment, the condom is a disposable device.

An insertable condom comprises a sheath of material formed to enclose a volume leaving an opening at one end, a ribbed member connected to the sheath of material, and an anchor point attached to the ribbed member. It will be understood by one skilled in the art that in one embodiment, such the insertable condom may be employed by a user separately from the thong device.

A wearable prophylactic system comprises a garment having a strap with an engagement device and an insertable condom. The insertable condom in turn comprises a sheath of material formed to enclose a volume leaving an opening at one end, a ribbed member connected to the sheath of material and an anchor point attached to the ribbed member. The anchor point is attachable to the engagement device of the strap to secure the insertable condom to the garment.

A method for manufacturing a prophylactic device comprises forming a first portion of a prophylactic device using a poured molding process and subjecting the first portion of the prophylactic device to a dip molding process to add a second portion to the prophylactic device.

The first portion comprises a base portion, at least one ribbed member connected to the base portion and an annular member connected to the at least one ribbed member. The second portion comprises a sheath of material formed to enclose a volume defined by the base portion and the annular member, leaving an opening at one end.

### BRIEF DESCRIPTION OF THE DRAWINGS

FIG. 1 shows an isometric view of a condom according to one embodiment of the present invention;

FIG. 2 shows another isometric view of the condom of FIG. 1.

FIG. 3 shows a side view of a condom according to one embodiment of the present invention;

FIG. 4 shows an axial view of a condom according to one embodiment of the present invention;

FIG. 5 is a detailed view of a portion of the condom of FIGS. 1-4 showing a snap hole disposed in a flange proximate to a ribbed member;

FIG. 6 shows a thong designed to secure to and prevent slippage of the insertable condom of one of FIGS. 1-5 when worn by a user.

FIG. 7 shows the thong of FIG. 6 after assembly

FIG. 8 shows one embodiment of the thong of the present invention being worn;

FIG. 9 shows a rear view of the thong of FIG. 8.

Before any embodiment of the invention is explained in detail, it is to be understood that the invention is not limited in its application to the details of construction and arrangements of components set forth in the following description, or illustrated in the drawings. The invention is capable of alternative embodiments and of being practiced or being carried out in various ways. Also, it is to be understood that the terminology used herein is for the purpose of illustrative description and should not be regarded as limiting.

### DETAILED DESCRIPTION OF THE INVENTION

The insertable condom device of this invention is best understood by reference to the attached drawings.

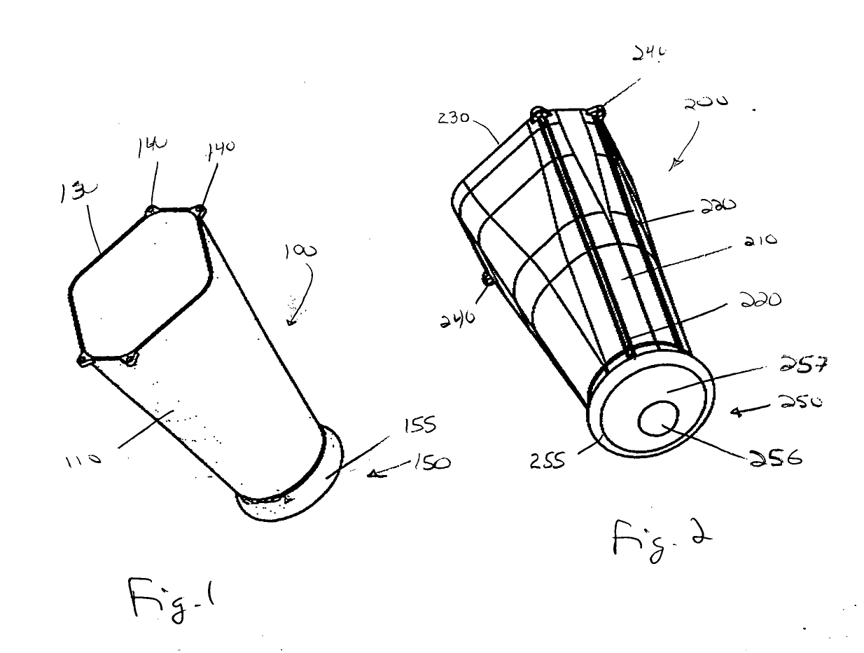

FIG. 1 shows an isometric view of a condom according to one embodiment of the present invention. A condom 100 comprising a basal aperture 130 and a tip 150 with a sheath 110 running therebetween. The basal aperture 130 is designed to accommodate the genitalia of a sexual partner of a wearer of the condom 100. The tip 150 of the condom 100 includes an annular member 155 to secure the condom 100 within the vaginal canal of a wearer. The condom 100 further comprises snap holes 140 which may be used to secure the condom 100 a separate garment.

FIG. 2 shows another isometric view of an embodiment of a condom according to the present invention. The condom 200 comprising a basal aperture 230 and a tip 250, with a sheath 210 running therebetween. In addition, a plurality of ribbed members 220 run along the surface of the sheath 210 further connecting the basal aperture 230 and the tip 250. These ribbed members 220 help secure the structure circumscribing the snap holes 240 to the condom 200.

The tip 250 of the condom 200 includes an annular member 255 as well as a teat 256 beneath which lies a reservoir 257. The tip 250, including the teat 256, defines and partially encloses a volume having an opening disposed towards the inside of the condom 200. The teat 256 forms a pinched portion of the tip 250 allowing for extra volume within the reservoir 257. The reservoir 257 is provided to contain the semen and bodily fluids of a partner of the wearer of the condom 200. As such, the tip 250 together with the condom 200 forms an enclosed watertight device for receiving these fluids, keeping them away from the surface of the vaginal canal in which the condom 200 is disposed.

The annular member 255 at the closed end of the condom 200 may be used to insert the condom 200, and holds the condom 200 in place inside the vagina sliding into place as it does behind the wearer's pubic bone. The basal aperture 230 at the open end of the condom 200 remains on the outside of the vagina during intercourse. In one alternative embodiment of the present invention, a portion of the open end of the condom 200 is designed to cover the wearer's vulva, further protecting the wearer's genitals.

Snap holes 240 are disposed proximate to one end of the ribbed members 220. The snap holes 240 allow the condom 200 to be secured to a thong, which will be discussed in further detail below with reference to FIGS. 6 through 9. When the condom 200 is secured to the thong at a discrete number of points defined by the snap holes 240, stresses transferred from the condom 200 to the thong during intercourse, especially stress along the long axis of the condom 200, is concentrated at these points. This can lead to tearing of the material of the condom 200 and a resulting failure of the device as a whole.

Accordingly, the structure of the condom 200 must be strong enough to withstand these stresses. On the other hand, it is also desirable that the condom 200 be as light and flexible as possible to transmit the sensation felt by the user during intercourse. The ribbed members 220 and the sheath 210 work together as a composite system to accomplish these goals. The ribbed members 220 provide strength to the structure circumscribing the snap holes 240 of the condom 200. The sheath 210 on the other hand, is thinner than the ribbed members 220. While not strong enough to provide a base to make a mechanical attachment to as the ribbed members 220 are, the sheath 210 provides a fluid barrier during sex while remaining thin enough to not significantly decrease the sensation of intercourse.

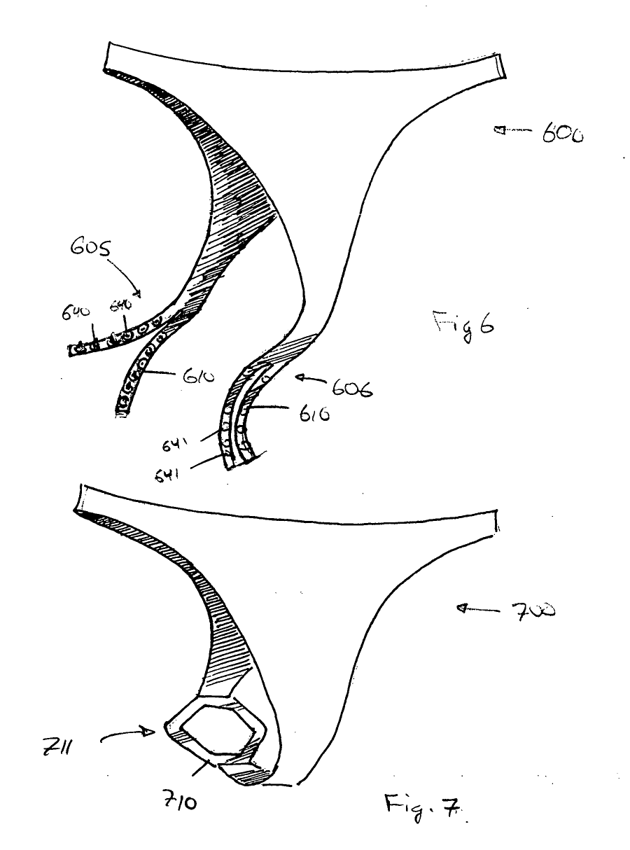

Turning now to FIG. 5, a detailed view of a portion of a condom is shown. FIG. 5 shows with greater clarity the intersection and interaction of a ribbed member 520 and a sheath 510 of a condom according to one embodiment of the present invention. The ribbed member 520 attaches to a flange 541 having a snap opening 540 disposed therein. While the material comprising the sheath 510 alone may not be a strong enough to secure the flange 541 to so that the snap hole 540 may be secured to a separate device, the thicker ribbed member 520 provides the additional strength necessary to accomplish this.

Turning again to FIG. 2 to discuss methods of manufacture of an exemplary embodiment of the present invention, the condom 200 may be manufactured using a two stage molding process. A first portion of the condom 200 is formed using a poured molding process. In one exemplary embodiment, this first portion comprises a ring shaped portion of material circumscribing the basal aperture 230, the ribbed members 220 and the annular member 255. This first portion may then be subjecting to a dip molding process to add a second portion to the condom 200. In one embodiment, the second portion comprises the sheath 210 and the tip 250.

The dip molding process allows the addition of the sheath 110 and tip 250 in the form of a thin skin over the underlying frame of the first portion, which may include the ribbed members as described above. If further shaping is thereafter required, in a further alternative embodiment, the condom 200 may be stretched over a second mold until the desired shape and thickness of the condom 200, and more particularly, the sheath 210, are reached.

In another alternative embodiment, the first portion comprises a ring shaped portion of material circumscribing the basal aperture 230, and one or more ribbed members 220. This first portion is then subjected to a dip molding process to add a sheath, so that a condom is formed originally without the annular member 155 or the tip 150. The sheath is fashioned with an open end meeting the portion of material circumscribing the basal aperture 230, and a closed end replacing the tip 150. An annular member may be added later as a separate element to a condom formed in this fashion.

In an exemplary embodiment, the condom 200 is made out of a durable latex material. In an alternative embodiment, the condom may made out of polyethylene or another appropriate material known to one skilled in the art.

FIG. 3 shows a side view of a condom according to one embodiment of the present invention. In this view the condom 300 is shown including a basal aperture 330, a tip 350, and a sheath 310 therebetween. As in previous embodiments, snap holes 340 are provided around the basal aperture 330. The tip 350 is shown including an annular member 355 and a teat 356 having a reservoir 357 beneath.

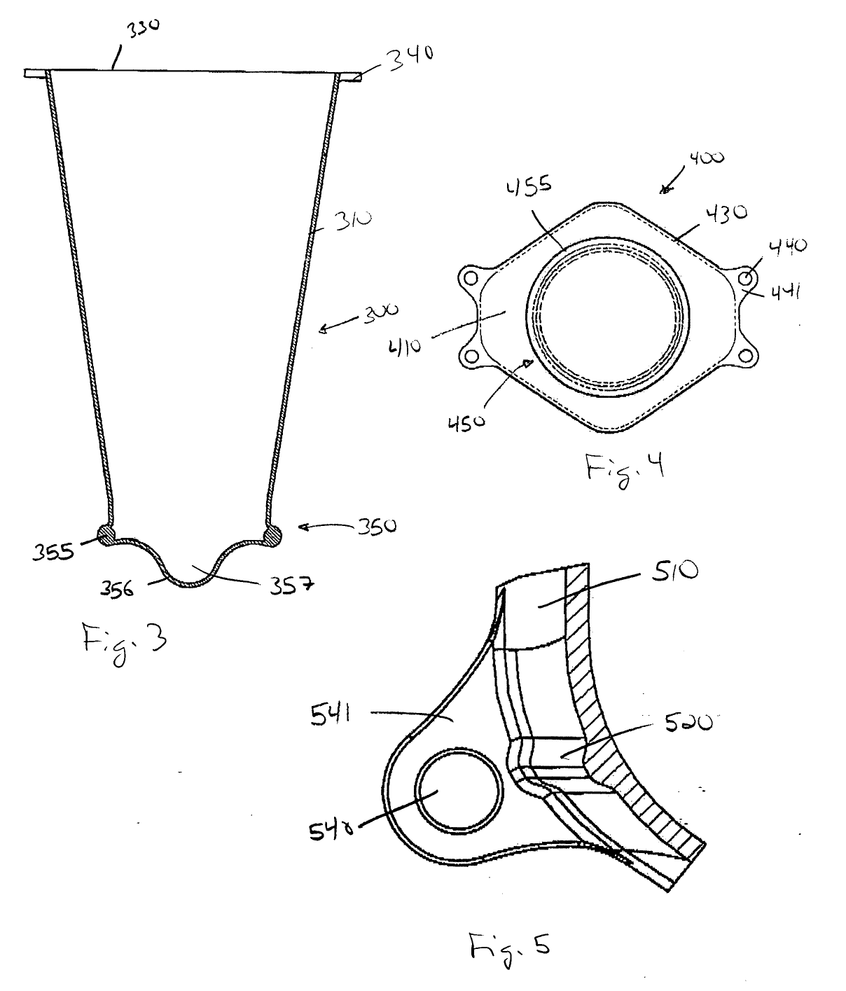

FIG. 4 shows an axial view of a condom according to one embodiment of the present invention. The condom 400 includes a basal aperture 430, a tip 450, and a sheath 410 therebetween. Again, as in previous embodiments, snap holes 440 are provided around the basal aperture 430, and the tip 450 is shown including an annular member 455. The basal aperture 430 is roughly hexagonal in shape. The longer axis of the basal aperture 430 is designed to align with the longer axis of the vaginal opening of the wearer of the of the condom 400, allowing for optimal placement and fixation of the condom 400 in the vaginal opening of the wearer. It will be understood by one skilled in the art that in alternative embodiments, the basal aperture 430 may be circular or ovoid in shape. It will also be understood that the number of flanges 441 and snap holes 440 may be altered. The condom 400 may be formed having anywhere from a single snap hole to a plurality greater than four. Furthermore, the shape of the flanges 441 surrounding the snap hole(s) may be varied, or the flanges 441 may be dispensed with entirely such that the snap hole(s) reside in a thickened portion of material circumscribing the basal aperture 230.

Furthermore, the basal aperture 430 of the condom 400 is designed to prevent the slippage of the condom 400 in the vaginal opening of the wearer whereby the entire condom 400 is pushed into the wearer's vagina during intercourse, frustrating the purpose of the device. The flanges 441 around the circumference of the basal aperture 430 act as barriers to hold the basal aperture 430 of the condom 400 over the vaginal opening of the wearer. In addition, the snap holes 440 are designed to interface with a thong type garment to further secure the condom 400 in place, and to prevent the movement of the basal aperture 430 across or into the vaginal opening of the wearer.

A thong type garment is shown in FIG. 6 designed to secure to and prevent slippage of the insertable condom of one of FIGS. 1-5 when worn by a user. Although a thong type garment is shown in FIG. 6, it will be understood by one skilled in the art that the thong 600 may be replaced with panties, panty girdles, pantyhose, G-strings, tights, and other female undergarments. In one embodiment, the thong 600 may be made from a stretchable, breathable, comfortable, and attractive material. In a further embodiment, the thong 600 is a reusable device. In yet another embodiment, the condom used with the thong 600 would be thrown out after a single use, such that the condom may be replaced repeatedly without having to discard the thong 600.

Because a common cause of failure of prior art insertable condoms was the slippage of the basal aperture of the condom into the vaginal opening of wearer, or the sideways slippage of the basal aperture of the condom such that the penis of the male partner of the wearer penetrates between the vaginal wall and the outside of the condom, the thong 600 is used with the insertable condom to help prevent such slippage. It accomplishes this by securing the basal opening of the condom fully over the vaginal opening of the wearer of the present invention.

To this end, the thong 600 is provided with a first strap pair 605 and a second strap pair 606, each having two straps 610 in the embodiment shown. The straps 610 of the first strap pair 605 are each provided with at least one first engagement device 640. Likewise, the straps 610 of the second strap pair 606 are each provided with at least one second engagement device 641.

In this manner the straps 610 of the first strap pair 605 and the second strap pair 606 may be connected to each other at different points allowing the thong 600 to fit women of different sizes. The snap holes 140 of the condom 100 of FIG. 1 may be secured between a first engagement device 640 and a second engagement device 641 so that the condom 100 is secured as a whole to the thong 600. It will be understood by one skilled in the art that various methods may be used to secure the snap holes shown in FIG. 1 to the engagement devices of FIG. 6. In one exemplary embodiment, the engagement devices may be metal snaps having a portion passing through the snap holes. In an alternative embodiment, the snap holes 140 of FIG. 1 may replaced with velcro fasteners that work with corresponding fasteners on the engagement devices of the straps to accomplish the same task. In another embodiment, a hook and eye system may be used.

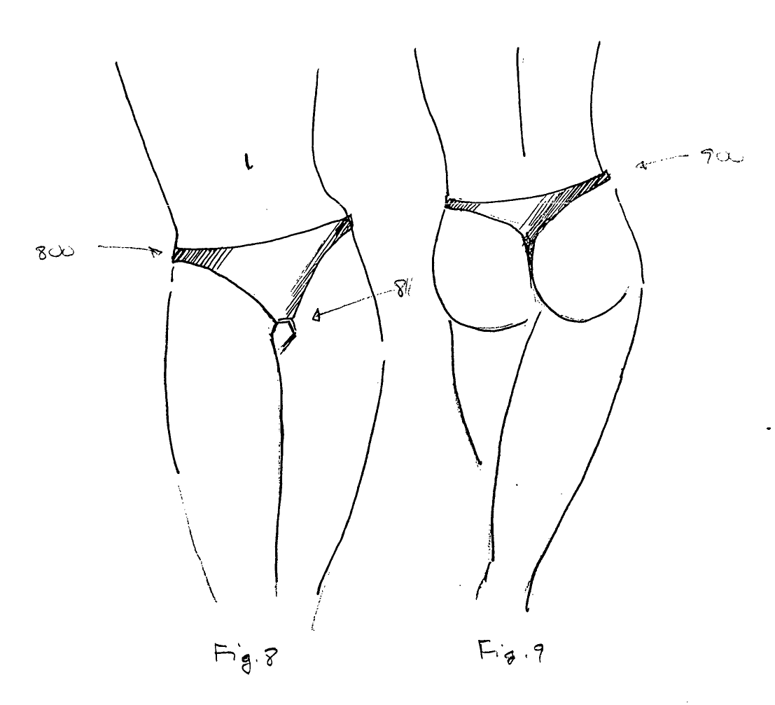

FIG. 7 shows a thong 700 of a design similar to that shown in FIG. 6, wherein the straps 710 have been attached to each other by their engagement devices to form a retaining aperture 711. An insertable condom may be secured in this aperture by positioning a first engagement device 640 and a second engagement device 641 of FIG. 6 one either side of each of the snap holes 440 of FIG. 4. The engagement devices thus pass through these snap holes to secure to each other, fixing the snaps holes and by extension, the flanges and the condom itself in place in the retaining aperture 711. This arrangement adds further stability to the position of an insertable condom, leading to a decrease in the incidence of failure of the device due to lateral or longitudinal slippage.

FIGS. 8 and 9 shows front and rear views, respectively, of the one embodiment of a thong being worn. FIG. 8 shows a retaining aperture 811 of the thong 800 positioned over the crotch of a wearer. The thong 800 may be assembled together with an insertable condom, and the combined assembly of the thong and condom may be worn beneath a user's clothing as if it were an ordinary undergarment.

Returning to FIG. 6, in an alternative embodiment the straps 610 of the first strap pair 605 and the second strap pair 606 may each be provided with a series of engagement devices. It will also be understood that the number of straps 610 may be altered without impinging significantly on the functionality of the device shown. For example. In an alternative embodiment, the thong 600 may be formed having a single pair of straps 610 only, which attach to a pair of snap holes on an insertable condom. In yet another alternative embodiment, the thong 600 may be formed with a plurality of straps that attach to the insertable condom in place of, rather than in addition to, attaching to each other. In this embodiment the snap holes of the insertable condom engage directly with a single engagement device of the thong 600 to secure the basal aperture of the insertable condom over the vaginal opening of the wearer.

1. An insertable condom comprising:
a sheath of material formed to enclose a volume leaving an opening at one end;
a ribbed member connected to the sheath of material; and
an anchor point attached to the ribbed member.

2. The insertable condom of claim 1 wherein the ribbed member is a reinforcing member to maintain the structural integrity of the insertable condom against forces applied at the anchor point.

3. The insertable condom of claim 1 wherein the ribbed member runs along the surface of the sheath of material.

4. The insertable condom of claim 1 wherein the ribbed member is formed integral with the sheath of material.

5. The insertable condom of claim 1 further comprising a tip attached to the sheath of material, wherein the tip has an annular member therein.

6. The insertable condom of claim 1 wherein the insertable condom is formed from latex.

7. The insertable condom of claim 1 wherein the insertable condom is formed from polyethylene.

8. The insertable condom of claim 1 having a plurality of ribbed members with a plurality of anchor points attached thereto.

9. A wearable prophylactic system comprising:
a garment having a strap with an engagement device; and
an insertable condom comprising:
a sheath of material formed to enclose a volume leaving an opening at one end;
a ribbed member connected to the sheath of material; and
an anchor point attached to the ribbed member;
wherein the anchor point is attachable to the engagement device of the strap to secure the insertable condom to the garment.

10. The wearable prophylactic system of claim 9 wherein the ribbed member is a reinforcing member to maintain the structural integrity of the insertable condom against forces applied to it at the anchor point.

11. The wearable prophylactic system of claim 9 wherein the garment further comprises a second strap with a second engagement device; and
wherein the anchor point attached to the ribbed member is attachable to both the engagement device of the strap as well as the second engagement device of the second strap.

12. The wearable prophylactic system of claim 11 wherein the engagement device and the second engagement device are attachable to each other.

13. The wearable prophylactic system of claim 12 wherein the garment further comprises a third strap with a third engagement device, and a fourth strap with a fourth engagement device; and
wherein the third engagement device and the fourth engagement device are attachable to each other such that the strap, second strap, third strap and fourth strap together form a retaining aperture for securing the insertable condom therein; and
wherein the anchor point of the insertable condom is attachable to at least one of the engagement device, the second engagement device, the third engagement device and the fourth engagement device to secure the insertable condom to the garment.

14. The wearable prophylactic system of claim 9 wherein the ribbed member runs along the surface of the sheath of material.

15. The wearable prophylactic system of claim 9 wherein the ribbed member is formed integral with the sheath of material.

16. The wearable prophylactic system of claim 9 wherein the insertable condom further comprises a tip attached to the sheath of material, wherein the tip has an annular member therein.

17. The wearable prophylactic system of claim 9 wherein the insertable condom is formed from latex.

18. The wearable prophylactic system of claim 9 wherein the insertable condom is formed from polyethylene.

19. The wearable prophylactic system of claim 9 having a plurality of ribbed members with a plurality of anchor points attached thereto.

20. A method for manufacturing a prophylactic device comprising:
forming a first portion of a prophylactic device using a poured molding process; and
subjecting the first portion of the prophylactic device to a dip molding process to add a second portion to the prophylactic device.

21. The method of claim 20 wherein the first portion comprises:
a base portion;
at least one ribbed member connected to the base portion; and
an annular member connected to the at least one ribbed member.

22. The method of claim 21 wherein the second portion comprises a sheath of material formed to enclose a volume defined by the base portion and the annular member, leaving an opening at one end.
